(19) United States
(12) Patent Application Publication
(10) Pub. No.: US 2006/0104958 A1
(43) Pub. Date: May 18, 2006

(54) Tissue engineered cardiac constructs

(76) Inventor: Robert E. Akins JR., Newark, DE (US)

Correspondence Address: MCGUIREWOODS, LLP, 1750 TYSONS BLVD, SUITE 1800, MCLEAN, VA, 22102, US

(21) Appl. No.: 11/265,515

(22) Filed: Nov. 3, 2005

Related U.S. Application Data
(63) Continuation of application No. 10/141,768, filed on May 10, 2002, now abandoned.
(60) Provisional application No. 60/290,026, filed on May 11, 2001.

(51) Int. Cl. A61K 35/34

(52) U.S. Cl. 424/93.7; 424/423

(57) ABSTRACT

Tissue-engineered bio-active cardiac constructs (BACCS) are described. These constructs are prepared by combining a suspension of isolated cells with a suitable support such as an extra-cellular filament or matrix. The isolated-cell component of the constructs is prepared from mammalian cardiac tissue, or from cells that differentiate into cardiac tissue. Extra-cellular filaments and/or matrices are prepared from naturally-occurring or synthetic polymers and may be biodegradable or non-biodegradable, depending on the application. Bio-active cardiac constructs are prepared in suspension culture using a defined, serum-free medium. Bio-active cardiac constructs are functionally and morphologically similar to intact cardiac tissue and may be used as tissue equivalents for research or drug testing applications, as well as in surgical applications for the treatment of heart disease.

This application is a continuation of and claims the benefit under 35 U.S.C. § 120 of U.S. application Ser. No. 10/141,768, filed May 10, 2002, which in turn is based on U.S. Provisional Application No. 60/290,026, filed on May 11, 2001, the contents of which are herein incorporated by reference in their entirety.

### FIELD OF THE INVENTION

This invention relates to tissue-engineered construction and more particularly to human bio-active cardiac constructs that have physiological characteristics of cardiac tissue.

### BACKGROUND AND SUMMARY OF THE INVENTION

Congenital and acquired heart diseases are substantial health problems. Congenital heart defects occur in nearly 14 of every 1,000 newborn children. Of all congenital defects, heart defects are the most common and are the leading cause of death in the first year of life. In addition to congenital defects, many people born with structurally normal hearts suffer cardiac malfunctions at some point during their lives due to disease, infection, or poor coronary circulation. According to U.S. government statistics, approximately 33% of all deaths are related to some type of heart disease.

Treatments for severe cardiac problems have become highly advanced, but when they fail, organ transplantation remains the only other option. Approximately 1,600 heart transplants are performed each year. Unfortunately, there is a profound shortage of donor hearts. It has been estimated that as many as 40,000 patients could benefit if heart implant material were available. Thus, there is a need for new approaches to treating sever cardiac problems. The possibility that cardiac function may be recovered through tissue engineering has great appeal. Implant materials that are produced in the lab by tissue engineering methods and used to augment or replace defective tissue could greatly alleviate the profound shortage of donor tissue organs.

Additionally, a large number of animals are used each year in research, drug testing, drug development, pharmacological testing, and education. Statistics are difficult to obtain, but the USDA does track laboratory animal use excluding rats and mice. In 1998, more than 1.2 million “non-rat/non-mouse” laboratory animals were used in the United States. Animal models are not always adequate representations of human biology, and engineered tissue equivalents could improve the physiological relevance of in vitro testing and reduce the number of animals required for research.

Cell culture alternatives to the use of intact animals and organs are routinely utilized for testing. However, typical cell culture models confine cells to a two-dimensional monolayer and a configuration that does not resemble the organization of cells within the intact tissue of interest. There is increasing evidence that cellular activities and responses are profoundly affected by three-dimensional organization, especially in the case of cardiac tissue. The development of a tissue material, which would be a tissue homologue that could mimic the physiological response of intact organs, would provide superior cell culture models for in vitro research, development, and testing.

Such a tissue material could reduce the number of animals required for testing and improve the physiological relevance of in vitro testing. Additionally, multiple tissue-equivalent materials could be prepared from a single donor organ. Further, the numerous species differences between humans and the animal models used for the research, development, and testing of cardio-active drugs and devices typically preclude the use of data derived from animal models to draw conclusions about the applicability to humans.

The use of human tissue-equivalents in vitro to test the safety, efficacy, and mechanism of action of potential therapeutic agents is clearly desirable, especially since current methods of analysis fall short [Owens, 2001#2956; lannini, 2001#2957]. In addition, the studying human cardiac-tissue equivalents could dramatically expand our understanding of cardiac cell biology, cardiac physiology, and the relationships between cardiac function and structure during embryogenesis and cardiac remodeling.

Accordingly, the present invention is directed to 1) providing a human bio-active cardiac construct (BACC), 2) a method of utilizing a BACC to augment cardiac tissue in a patient, and 3) a method of utilizing a BACC for assessing the pharmacological activity of test compounds.

An objective of the present invention is to provide a human bio-active cardiac construct from cells suitable for use in forming cardiac tissue material on at least one support where the cells are organized on the support to provide a three-dimensional structure having physiological characteristics of human cardiac tissue.

The types of cells that may be used in forming the human bio-active cardiac construct may include, cardiomyocytes, endocardial cells, cardiac adrenergic cells, cardiac fibroblasts, vascular endothelial cells, smooth muscle cells, cardiac progenitor cells, and stem cells. Depending on the application of the human bio-active cardiac construct and the type of cardiac tissue material that is desired, the above types of cells may be used independently or combined with one another.

Materials that are suitable for supports include sutures, meshes, foams, gels, ceramics, acellularized extra-cellular matrix material, and the like. If a suture material is used for the support, the suture material is preferably made of silk, polypropylene, polyamide, polyvinylidene, polyester, polyether, polydioxanone, nylon, linen, cotton, plain gut, chromic gut, poliglecaprone, polyglactin, polylactide, collagen, or naturally occurring protein, and the like, and may include a combination of these materials.

A further objective of the present invention is to provide a method for the augmentation of cardiac tissue. The method of the invention includes obtaining at least one human cardiac construct having cells organized on a support to provide a three-dimensional cell structure having physiological characteristics of cardiac tissue and implanting the human cardiac construct in a patient. The method may further comprise implanting a compound such as compounds that enhance vascularization, compounds that enhance cell survival, compounds that enhance cell proliferation, compounds that enhance cell differentiation, compounds that enhance tissue formation, compounds that inhibit fibrosis, compounds that inhibit inflammation, compounds that inhibit de-differentiation, and compounds that inhibit tumorigenesis.

A further objective of the present invention is to provide a method for analysis of pharmacologically active compounds. The method of the invention includes obtaining a cardiac construct having cells organized on a support to provide a three-dimensional cell structure having physiological characteristics of cardiac tissue, obtaining a test compound, introducing the test compound to the cardiac construct, and observing the response of the cardiac construct. This method may further comprise attaching at least one cardiac construct to at least one measuring device such as a strain gage. When a cardiac construct is attached to, for example, a strain gage, compounds such as compounds that have a chronotropic effect and compounds that have an inotropic effect may be tested. Alternatively, the method of the invention may comprise obtaining a cardiac construct, obtaining a test compound, introducing the test compound to the cardiac construct; adding a dye to the cardiac construct and observing the response of the cardiac construct. This method may further comprise observing responses by a detection instrument sensitive to the dye and selection of test compounds such as compounds that affect conduction, compounds that affect gene expression, compounds that affect cellular organization, and compounds that affect multicellular organization.

### BRIEF DESCRIPTION OF THE FIGURES

FIG. 1 illustrates a cross-sectional representation of a bio-active cardiac construct in accordance with one embodiment of the present invention showing critical features of the bio-active cardiac construct;

FIG. 2(a) illustrates a kit containing a plurality of bio-active cardiac constructs for testing or tissue augmentation in accordance with one embodiment of the present invention; (b) illustrates one bio-active cardiac construct attached to a frame.

FIG. 3 is a graph showing the effect of adrenergic drugs on Bio-Active-Cardiac-Construct contractile fibers.

### DETAILED DESCRIPTION OF THE INVENTION

The present invention is directed to a bio-active cardiac construct that may be implanted in to a patient to augment cardiac tissue or may be used for in vitro testing. Bio-active cardiac constructs are tissue engineered assemblages of cells that exhibit the three-dimensional organization of intact cardiac tissue, and that exhibit physiological functions and responses of intact cardiac tissue.

As discussed by Akins et al. (Tissue Engineering, 1999. 5(2): pp 103-18) and Akins and Sefton (New Surgery, 2001. 1(1): pp 26-32), human bio-active cardiac constructs share critical structural and functional characteristics with intact cardiac tissue, such as distinctive multi-cellular organization and oriented contractile function. This similarity allows for the potential use of a bio-active cardiac construct to augment cardiac tissue in a patient. Further, this similarity allows for the in vitro testing of pharmacologically active compounds on the bio-active cardiac construct to determine the effect of the compound on cardiac tissue. In substantial contrast, traditional cardiac cell cultures typically result in a two-dimensional monolayer of cells which do not exhibit the three-dimensional organization of cardiac tissue or the physiologic functions and responses of cardiac tissue.

The human bio-active cardiac constructs of the present invention are pieces of tissue that have elastic, tensile, and contractile properties similar to pieces of normal cardiac tissue. The cardiac construct can be manipulated by using surgical instruments just as pieces of normal cardiac tissue can be manipulated.

The three dimensional interactions among various cells, as well as those between cells and their external environment, affect physiological function in a variety of systems. Marx et al. (Journal of Clinical Investigation, 1994. 93(1): p. 131-9) have shown that the spatial organization of the surrounding cell matrix affected microvascular-endothelial-cell (MVEC) differentiation and the regulation of platelet-derived growth factor receptors. Sankar, S., et al. (Journal of Clinical Investigation, 1996. 97(6): p. 1436-46.) further suggested that differences in matrix conformation between 2D cultures and 3D-collagen-gel-based cultures were associated with the modulation of TGFβ receptors in MVECs. In human mammary epithelial cells, Huguet et al. (Journal of Biological Chemistry, 1995. 270(21): p. 12851-6.) found that Wnt5a gene expression was down-regulated during transitions from growth on 2D surfaces to growth in 3D gels. Kitamura, et al. (American Journal of Physiology, 1996. 270(4 Pt 2)) noted that mesangial cells cultured in nodules exhibited a phenotype that differed from that of cells cultured in 2D. In addition, the morphology of a variety of cells (including rat tendon fibroblasts, baby hamster kidney cells, macrophage-like cells, and neurons from rat dorsal root ganglia) have been found to be affected by the conformation of the fibronectin-coated surface on which the cells were grown.

The configuration and orientation of the cells is related to cell distribution and in the case of a bio-active cardiac construct is a function of the position of the cells just after adhesion and the subsequent migration and remodeling of the construct. Thus, in order to produce a culture that has tissue-like functions and properties, the initial conditions of the culture must be carefully selected. Configuration and orientation is further complicated by the fact that distribution of cells within a three-dimensional construct likely changes over time.

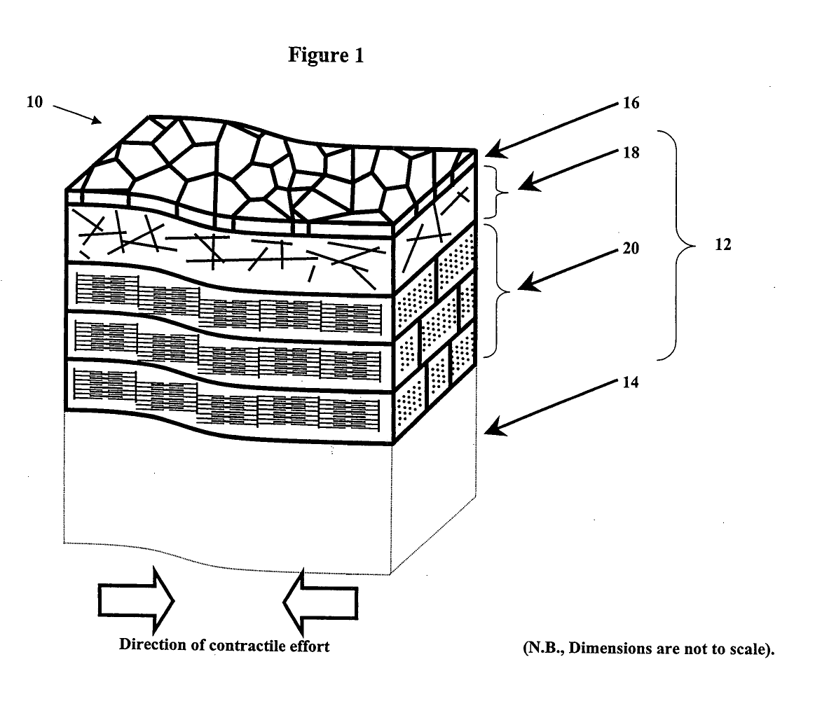

With reference now to FIG. 1, there is shown a portion of a bio-active cardiac construct 10 in accordance with one embodiment of the present invention. The bio-active cardiac construct 10 has multiple-layers of cells 12 arranged on a support 14. The multiple-layers are configured to resemble cardiac tissue and include a layer of endothelial cells 16 which may include endocardial cells, a layer of expressed matrix 18, and a layer of oriented myocardial cells 20.

Hence, a strategy to configure and orient the cell assemblages is an important component in developing a tissue identical culture. Any of three categories of strategies may be used. These include, but are not limited to: 1) the use of synthetic supports or scaffolds, 2) the use of natural support matrices, and 3) the free assembly of cells in fluid media. The use of scaffolds or matrices as supports may offer the advantage of producing a construct that is more amenable to manipulation for an end use such as implantation.

The support provides a stabilizing surface to which the cells can attach, grow, and organize into three-dimensional structures representative of cardiac tissue. The types of materials that may be used as supports is not limited except that the support should be compatible with the types of cells that are being used and should not interfere with the growth or organization of the cells to form cardiac tissue material. When the human bio-active cardiac construct is to be implanted in a patient, the material should be biologically compatible for implantation into a human.

Accordingly, selection of a suitable support in composition and form is important. In the selection of a support material, several characteristics are considered. Among these are biocompatibility with the environment in which the construct will be used, biodegradability, tensile strength, flexibility, elasticity, and cost. Suitable supports include, but are not limited to a mesh, foam, gel, ceramic, suture, or acellularized extra-cellular matrix material. Any suture material suitable for medical use may be used. Examples of suitable suture materials include, but are not limited to, silk, polypropylene, polyamide, polyester, polyvinylidene, polyester, nylon, linen, cotton, plain gut, chromic gut, poliglecaprone, polyglactin, polylactide, collagen, other type of suture material or fibrous polymer, or a combination of these. In one embodiment, a polyglactin suture material is used.

The dimensions of the human bio-active cardiac construct can be varied depending on the desired application and the size and shape of the support that is used. The dimensions will logically depend on the size and geometry of the support material that is utilized, since the cells that make up the bio-active cardiac construct initially adhere to the support and multiply over its surface.

Cells are needed for the production of human bio-active cardiac constructs. The minimum requirement for the cells is that they either have a native capacity for differentiation into human cardiac tissue or that the cells may be manipulated into forming human cardiac tissue. Accordingly, small samples of autologous or heterologous donor cardiac cells may be used for constructs. Alternatively, cells such as non-immunogenic universal donor cell lines, or stem cells can be used so long as they can be manipulated to form human cardiac tissue.

One source of cells is heterologous cells from donor organs. In this case, donor tissue from individual biopsies, surgical remnants, or whole donor organs could be used as cell sources for the preparation of implants. One benefit to the use of heterologous implants is that a single donor may provide constructs for many recipients, thus decreasing the demand for donor organs.

Autologous cells may also be utilized in the practice of one embodiment of the instant invention. For example, cells from a healthy section of a heart that has been damaged may be removed and grown in culture to form a human bio-active cardiac construct and then reintroduced to the damaged area of the autologous heart via implantation.

Cell types used may include, but are not limited to, cardiomyocytes, endocardial cells, cardiac adrenergic cells, cardiac fibroblasts, vascular endothelial cells, smooth muscle cells, stem cells, cardiac progenitor cells, and myocardial precursor cells. Depending on the application of the human bio-active cardiac construct and the type of cardiac tissue material that is desired, the above types of cells may be used independently or combined with one another. In one embodiment, bio-active cardiac constructs are composed of primary tissue isolates from the heart.

The cellular composition of the human bio-active cardiac constructs may be varied by controlling the process of assembly. In some embodiments, the cardiac construct is made up of myocytes cultured alone. In other embodiments, the human bio-active cardiac constructs may be made up of myocytes cultured in conjunction with other cell types. For example, human bio-active cardiac constructs comprised of co-cultured myocardial cells, endocardial cells, vascular smooth muscle cells, vascular endothelium, fibroblast, and adrenergic cells, or various subsets of those cell types, may be cultured together into an organotypic structure resembling cardiac tissue.

A human bio-active cardiac construct may be constructed of human or other animal cells, or a mixture of human and other animal cells. Further, the cells which are utilized in the formation of a BACC may be genetically engineered to, for example, contain a gene of interest such a gene expressing a growth factor.

The selection of cells to use in the construction of a Bio-Active-Cardiac-Construct is predicated by the desired use of the construct. For example, in one embodiment of a human bio-active cardiac suture for use in pharmacological testing, all of the cell types typically associated with the heart may be used to form a bio-active cardiac construct.

In one embodiment, a human bio-active cardiac construct includes an assemblage of cells grown on a suture material. Such a cardiac construct may be referred to as a bio-active cardiac suture, or BACS, and is suitable for attachment to a strain gage for testing, or for implantation into a heart during surgery.

On macroscopic observation, the bio-active cardiac suture has the appearance of a suture coated with biological material with an outer diameter of about 0.5 mm, and more preferably of about 0.3 mm. The length of a bio-active cardiac suture is determined by the length of the suture material that is utilized as the support. In one embodiment, the length of the bio-active cardiac suture may range from about 20 mm to about 30 mm, and in one embodiment is about 25 mm.

On microscopic observation, the biological material on the suture support of the bio-active cardiac suture has the appearance of cardiac tissue. Microphotographs have shown that the bio-active cardiac suture consists of a three-dimensional arrangement of cells layered on the surface of a supporting VICRYL suture (Ethicon, Inc., Somerville, N.J.). Scanning electron microscopy shows that a layer of endocardium surrounds the suture, and transmission electron microscopy indicates that the bio-active cardiac suture has a layered organization similar to cardiac tissue found in the heart.

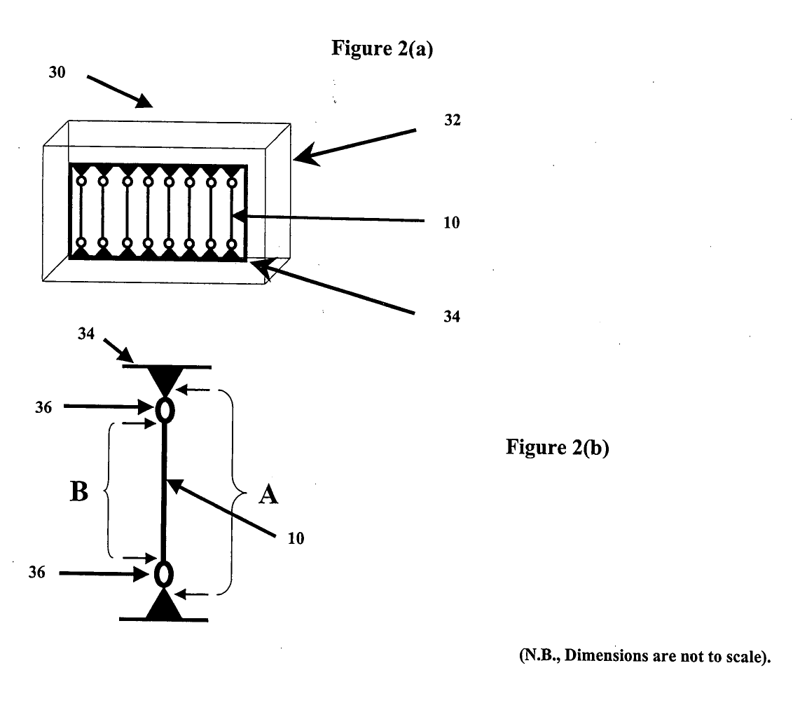

The utility of Bio-Active-Cardiac-Constructs lies in two areas: the treatment of heart diseases and the assessment of cardio-active compounds in research, development, and pharmacological testing. In the treatment of heart disease, implantation of a construct(s) provides for augmentation of diseased tissue. In the assessment of cardio-active compounds, constructs provide an improved in vitro model for testing. To this end, a kit containing a bio-active cardiac construct may be provided. FIG. 2 shows a kit 30 containing a plurality of bio-active cardiac constructs 10 in a container 32.

The container 32 is preferable composed of a material having a low biologic effect, but allows for gas diffusion such as fluoroethylene polymer or similar materials. In one embodiment, the container may be transparent allowing for visible inspection of the cardiac constructs.

Preferably, the container or packaging is filled with a stabilizing and nutritive medium that sustains the biological function and sterility of the cardiac constructs during storage and transit. Suitable media has been discussed above. In one embodiment, it is preferable to have as small a head space in the container as possible. Most preferably there would be zero head space in the container.

The container may contain one or more cardiac constructs. The number of constructs in a container is not particularly limited. Preferably, as shown in FIG. 2(a), they are aligned in one or more rows, parallel with one another to make their location and removal easier. A frame 34 may be utilized to hold the cardiac constructs in the container 32. Each cardiac construct 10 may be provided with a loop 36 on each end of the construct. These loops are useful for attaching the construct to various instruments such as a strain gage. If a construct with loops is desired, the construct would be removed from the frame by cutting at position A, as shown in FIG. 2(b). To provide a construct for implantation, the construct may be removed from the frame by cutting at position B.

Bio-active cardiac constructs may be implanted directly into, for example, damaged (or other) areas of a heart during heart surgery in order to augment contractile function. In one embodiment, heart disease would be treated by surgically placing one or more bio-active cardiac constructs across a non-functional region of the heart bridging to two healthy regions. The suture would be placed along the axis of contraction for that region of the heart. Such a placement would help to re-establish the correct propagation of the contractile signal and would provide contractile elements. In some cases, the implant material may include other compounds to improve vascularization or integration (e.g., VEGF).

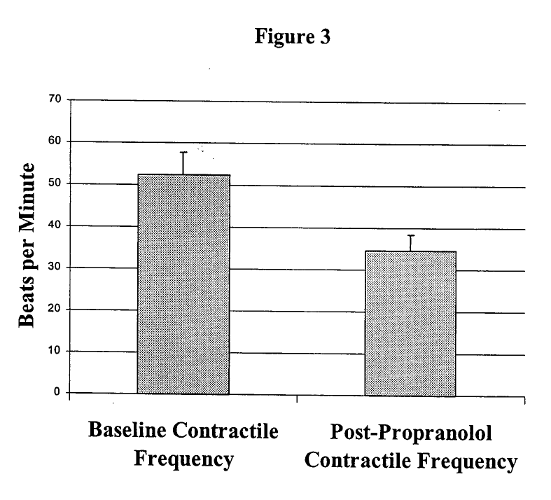

As mentioned above, bio-active cardiac constructs can serve as an improved in vitro model for the testing of pharmacologically active compounds, and the like. Pharmacologically active compounds could be tested by adding the compound to the construct and assessing the resulting effects on various properties of the bio-active cardiac construct. The effect of the compound on contractility, cellular or multi-cellular organization, metabolic activity, or gene expression may be tested. In one possible use, a linear arrangement of cells grown on a suture may be attached to a strain gage and perfused with compounds that are suspected to cause changes in spontaneous or induced contractile activity. The effect of the compound would be monitored over time by recording the periodicity and strength of subsequent contractions. As an example, propranolol added to spontaneously contractile constructs of rat tissue resulted in a decrease in contractile frequency, as shown in FIG. 3. Propranolol has negative chronotropic effects on cardiac muscle. The spontaneous beat frequency was determined for each of six bio-active cardiac constructs before and after the addition of 2 μM propranolol. The drug treatment resulted in a 34% decrease in beat frequency. This decrease was significant (p<0.05, Student's t-test). The dose responsiveness of cardiac tissue to other adrenergic agonists and antagonists (or to other modulators or potential modulators of contractile activity) can be studied in a similar fashion using bio-active cardiac constructs.

To prepare bio-active cardiac constructs with functional and morphological similarity to intact cardiac tissue, disaggregated cells are combined with a support and a nutritive medium in a bioreactor. The methods for preparing the bio-active constructs utilize the capacity of isolated cardiac cells to re-establish complex, three-dimensional, cardiac organization in vitro. The method encourages the intimate cell:cell interactions seen in intact cardiac tissue from the earliest phases of culture. To this end, constructs are prepared in a low-shear suspension culture environment that allows the co-localization of cells in three dimensions for extended periods of time. Cells are inoculated into this environment at a relatively high density to promote cell:cell interactions. The cells should be introduced to the bioreactor in sufficient density to promote cell:cell interactions and growth required to establish complex, three-dimensional cardiac organization. The density of the cells will vary depending on certain variables such as, the exact type of cardiac tissue desired and the size and shape of the support. Generally, for most applications such as that for cardiac tissue on a suture support, a cell density ranging from about 0.5×10⁶ cells per cm² of support material surface area in 1 cc of medium to about 2.5×10⁶ cells per cm² of support material surface area in 1 cc of nutritive medium will be appropriate. The cells are preferably introduced into the bioreactor as a suspension. Any suspension medium known to those skilled in the art may be used.

The cells or cell suspension are combined in a nutritive medium. There are numerous nutritive media available for mammalian cell culture. The choice of medium for use in tissue engineering experiments must be made based on a variety of considerations. Media have profound effects on the proliferation, growth, and function of cells, and the medium chosen can dramatically alter cell phenotype in culture. Typically, basal culture media contain essential nutrients, trace elements, vitamins, lipids, electrolytes, and sources of energy. In addition, some type of additive is generally added to a basal medium prior to its use. The most commonly used addition is sera from fetal, newborn, or adult cows. Although basal media can be very specifically defined, the serum itself is not a defined component, and there is considerable variability in sera from different sources or in different lots of serum from the same source. Preferably, a serum-free medium, in which the additives to the medium are defined, is used in forming bio-active constructs of the present invention.

For tissue engineering, it is important to reproducibly generate implantable constructs. The use of a serum-free medium allows the investigator to specifically control the levels of growth factors, cytokines, and functional modulators present at any time during the culture and avoids stringent, and expensive, quality-control testing of the medium to screen for infectious agents which may be present in serum. The use of serum-containing media is not precluded in the current method but as a matter of convenience and control, serum-free media is preferred.

Serum-free media have been described in the literature for the culture of mammalian cardiac cells and are known to those skilled in the art. Typically, these formulations are basal media that are supplemented with specific hormones or factors like insulin, thyroid hormone, holo-transferrin and steroids. For specific examples, see Karliner, J. S., et al., Biochemical and Biophysical Research Communications, 1985. 128(1): p. 376-382; Libby, P., Journal of Molecular & Cellular Cardiology, 1984. 16(9): p. 803-811; Kessler-Icekson, G., et al., Experimental Cell Research, 1984. 155(1): p. 113-120; Mohamed, S. N., R. Holmes, and C. R. Hartzell, In Vitro Cell and Developmental Biology, 1983. 19(6): p. 471-8; and Freerksen, D. L., N. A. Schroedl, and C. R. Hartzell, Journal of Cellular Physiology, 1984. 120(2): p. 126-34, each specifically incorporated herein by reference in their entireties.

Alternatively the medium may involve combinations of serum-free medium and fibroblast-conditioned medium for cardiac cell culture such as those described in Suzuki, T., H. Hoshi, and Y. Mitsui, FEBS Letters, 1990. 268(1): p. 149-151; Suzuki, T., et al., Journal of Molecular and Cellular Cardiology, 1997. 29(8): p. 2087-2093; and Suzuki T., et al. Journal of Cardiovascular Pharmacology, 1991. 17(Suppl 7): p. S182-186, each specifically incorporated herein by reference in their entireties. Any of these media can be used for cardiac tissue engineering using the current method. For example, serum-free and hormone-free medium (SF—HF) such as that described by Mohamed, S. N., R. Holmes, and C. R. Hartzell, In Vitro Cell and Developmental Biology, 1983. 19(6): p. 471-8 specifically incorporated herein by reference in its entirety. SF—HF medium comprises a 1:1 mixture of DMEM and F12 with 1× MEM nonessential amino acids, 0.1× MEM vitamin solution, 0.025% fetuin, 1% BSA, 0.02 mg/ml ascorbic acid, 0.0.8 mg/ml additional CaCl₂, 100 μU/ml penicillin, 100 μg/ml streptomycin, 5 μg/ml insulin, 7.5 μg/ml transferrin, 5 ng/ml selenium, and 0.012% BSA-palmitate complex. Preferably, a serum-free hormone-supplemented medium (SFHM) such as that described by Freerksen, D. L., N. A. Schroedl, and C. R. Hartzell, Journal of Cellular Physiology, 1984. 120(2): p. 126-34 specifically incorporated herein by reference in its entirety, can be used for the culture of cardiac cells and the formation of Bio-Active-Cardiac-Constructs. SFHM comprises a 1:1 mixture of glucose-free/glutamine-free DMEM and F12 with 2.5 mg/ml sodium bicarbonate, 1× MEM nonessential amino acids, 0.1× MEM vitamin solution, 5 mM glucose, 0.025% fetuin, 1% BSA, 0.02 mg/ml ascorbic acid, 0.0.8 mg/ml additional CaCl₂, 100 μU/ml penicillin, 100 μg/ml streptomycin, 50 μU/ml insulin, 50 ng/ml hydrocortisone, 10 ng/ml EGF, 75 pg/ml T₃, 5.5 mM transferrin, 10 ng/ml selenium, and 0.012% BSA-palmitate complex. Other, suitable commercially available serum-free media that may be used with the present invention include, but is not limited to, SM3, and AI-1 Medium (KD Medical Company, Columbia, Md.), and other similar serum-free media.

The cells, support, and media are combined in a bioreactor. Many types of bioreactors have been conceived for cell culture. Traditional tissue culture methods are unsuitable for the production of cardiac implant materials as they constrain cells to proliferate and differentiate on 2D surfaces. The mosaic patterns produced only remotely resemble the organization seen in vivo. To grow cardiac tissue and produce a bio-active cardiac construct, specific bioreactor attributes should be considered. The considerations include the capability to mimic the mechanical and humoral environments of the tissue. A bioreactor should be able to encourage the formation and maintenance of cardiac tissue architecture. Of particular concern in cardiac tissue engineering is the minimization of damaging fluid shear and the co-localization of cells in a fluid environment for the extended periods needed to form intricate cell: cell associations.

Cardiac cells are exquisitely sensitive to mechanical stimulation. Accordingly, it is important that the bioreactor provide a low-shear environment. Bioreactors for 3D culture include high-shear spinner-flasks or the use of gel matrices and low-shear clinostats like the NASA-designed vessels (e.g., High Aspect Ratio Vessel, HARV; Slow Turning Lateral Vessel, STLV, Hydrodynamic-Focusing Bioreactor, HFB) or clinostatically rotated FEP-bags. The low-shear environment found in NASA bioreactors (<0.52 dyne/cm² for the HARV) and in rotated FEP bags are especially suitable for the culture of Bio-Active-Cardiac-Constructs.

The sedimentation of cells (or support material, etc.) in the bioreactor combined with the rotational movement of the medium results in the cell taking an elliptical path relative to the observer. By adjusting the rotation rate of the bioreactor, the dimensions of this elliptical path can be reduced so that the vessel, medium, and contents appear to maintain their relative relationships in a low-shear environment.

As discussed above, any of several bioreactors are suitable for use in the practice of this invention. In one embodiment of the current invention, a simple bioreactor comprising a VueLife fluoropolymer (PTFE) bag (American Fluoroseal Corporation, Gaithersburg, Md.), which is clinostatically rotated is used. VueLife bags are single 7 ml units with a single port to access the contents. The bags are optically transparent and the contents can be visualized on a standard light microscope without sampling. The PTFE material itself is gas permeable, and the entire bag serves as a gas exchange surface. When assembled and loaded, a VueLife bag is a closed, fluid-filled system that can be rotated to suspend the contents.

In other embodiments, a NASA-designed rotating wall bioreactor is used. The use of these bioreactors has been described in detail by the applicants (Akins, et al., In Vitro Cell and Developmental Biology—Animal 1997, 33: pp 337-343; Akins, R. E., et al., Tissue Engineering 1999, 5(2): pp 103-18; Akins, R. E. in Medizinische Regeneration und Tissue Engineering. EcoMed Publishing, Landsberg, Germany, 2000, Chapter XI-4: pp 1-16; Akins, R. E. in Methods of Tissue Engineering. Academic Press, San Diego: pp 915-925). Prior to use, the bioreactors are cleaned using distilled water and sterilized using ethylene oxide gas or a 70% (v/v) ethanol solution. Once assembled and filled, a rotating wall bioreactor is a closed, fluid-filled system that can be rotated to suspend the contents. They are loaded with support matrix and cell suspension and rotated at a rate to keep the matrix and cells in a state of static suspension relative to the walls of the vessel. The exchange of medium, addition of compounds, and sampling of the vessels is carried out through two access ports.

The cells that are added to the bioreactor are attachment dependent and contact inhibited. To initiate cell culture in the preferred embodiment of the invention, surfaces onto which the cells can attach may be provided. Standard support surfaces include, for example, Biosilon microcarrier beads (Nunc/InterMed, Roskilde, Denmark), CytoDex microcarrier beads (Sigma Chemical Company, ST. Louis, Mo.), non-oriented collagen cultispheres (HyClone Laboratories, Logan, Utah), oriented-collagen sutures (Organogenesis, Canton, Mass.), and various other suture materials (e.g., polyglycolic acid, Davis & Geck, Manati, PR). If not shipped sterile, these support surfaces must be sterilized prior to use.

Further, if a serum-free medium is to be used, to encourage attachment of the cells to the support, the support is preferably coated with fibronectin (or laminin or collagen) prior to culture. Support surfaces are provided to the cells in order to initiate the organizational process and to orient the cellular assembly.

The bioreactor is placed into an incubator with a controlled environment. The composition of the gas environment in the incubator is predicated by the selection of medium. In a preferred embodiment, a mixture of air with 5% CO₂ is used in conjunction with bicarbonate-buffered medium. Alternatively, air alone has been used in conjunction with a HEPES-buffered medium. The temperature of the incubator environment is held at 37 degrees Celsius, and the relative humidity of the incubator environment is held at 90%.

One embodiment utilizes primary human cardiac cells; a biodegradable support scaffold which are pieces of fibronectin-coated polyglactide suture (VICRYL, Ethicon, Inc., Somerville, N.J.); a serum-free medium (AI-1 medium, KD Medical, Columbia, Md.); and a bioreactor of a clinostatically rotating bag constructed of an enert polymer, for example VueLife bags, (American Fluoroseal Company) which are constructed of polytetrafluoroethylene (PTFE) polymer, i.e. Teflon®.

The progress of the culture may be monitored using a variety of tools. Small amounts of medium may be analyzed for the presence of metabolites and waste products. As indicators of metabolic activity, changes in the level of nutrients may be monitored. Samples of constructs grown in bioreactors may be assessed non-invasively for spontaneous or stimulated contractile activity. Small pieces of constructs may be collected for the analysis of metabolic enzyme activities, gene expression profile, electrical signal propagation, or for differentiation status as indicated by histomorphology and fluorimetric staining.

Once the culture has progressed enough to exhibit the three-dimensional cellular organization of cardiac tissue over a significant portion of the substrate, the bio-active cardiac construct is basically complete. One skilled in the art will recognize that incubation times and parameters will vary depending on the cells, medium, and support that is selected. Such parameters are within routine experimentation.

The following examples are provided as illustrative examples of certain embodiments of the present invention and should not be viewed as limitations of the present invention.

### EXAMPLES

### Example 1

### Preparation of Cells

Materials and Reagents Required in one embodiment include: Tissue; magnetic stir plate; water-jacketed spinner flask, sterile, 25-50 ml hooked up to a circulating water bath set at 36 (±0.5)° C.; 100 mm bacteriological grade culture dishes; sterile dissecting instruments (forceps and scissors); sterile Ca²⁺—Mg²⁺-free Hanks Balanced Salt Solution (HBSS) buffered with 10 mM HEPES to pH 7.4 (HBSSH); AI-1 medium (K.D. Medical, Columbia, Md.); 70% ethanol; two 250 ml beakers plus a squeeze bottle; proteolytic enzyme solution prepared in HBSSH to contain Trypsin (Worthington Biochemical Corporation, Freehold, N.J.) at a total activity of 238 Upper 100 ml, Chymotrypsin (Worthington Biochemical Corporation, Freehold, N.J.) at a total activity of 278 Upper 100 ml, and Elastase (Calzyme Laboratories, San Luis Obispo, Calif.) at a total activity of 5.32 Upper 100 ml; sterile cotton balls or small gauze pads; sterile 50 ml centrifuge tubes; polyglactide sutires (Ethicon, Sommerville, N.J.) precoated the previous day with fibronectin; sterile pipets, 1, 10, and 25 ml; hemocytometer or other cell counter; centrifuge for spinning 50 ml tubes; Falcon cell strainers (Becton Dickinson, Franklin Lakes, N.J.). Procedure for preparing cardiac cells according to one embodiment.

100 mm culture dishes were filled with ˜25 ml of sterile HBSSH and fragments of tissue to be used were placed into the HBSSH using sterile techniques. Each fragment was examined and any pulmonary or other non-cardiac tissue that was present was removed. The tissue was transferred to a clean, dry 100 mm dish, and minced into ˜1-2 mm3 pieces. The minced tissue was transferred to the water-jacketed spinner flask. After the minced tissue was placed in the flask, the remainder of the sterile HBSSH was added to the tissue to rinse away excess blood cells and debris and the rinse solution was decanted leaving the tissue fragments in the flask. Proteolytic enzyme solution was added to the minced tissue at a ratio of 2:1 (i.e., 2 volumes of enzyme per volume of tissue). The flask was equipped with a stirrer and positioned on a magnetic stir plate with the rate of revolution set to just suspend the fragments (<200 rpm). The solution and tissue were incubated for 15 min., then the enzyme solution was decanted into a waste beaker. This step was repeated 1 time. Proteolytic enzyme solution was added to the minced tissue; using 1 ml per heart tissue equivalent. The solution was stirred to suspend the fragments and incubated for 20 minutes. After incubation, the enzyme solution was decanted into a 50 ml centrifuge tube containing 15 ml of growth medium. The cells were collected by gentle centrifugation at ˜250×g for 10 min. After centrifugation was complete, the solution was decanted from the pellet into the waste beaker with care not to dislodge the soft pellet. The pellet was resuspended in 5 ml of growth medium. This step was repeated until no more cells come out of the tissue fragments (i.e., the enzyme solution was clear after the 20 minute incubation).

The collected cells were pooled and gently triturated to resuspend the cell suspension. This suspension should be strained through cell strainers to remove any undissociated chunks of tissue. Trypan-Blue excluding cells were counted using a hemocytometer. The cells were then diluted with growth medium to a density of 1×10⁶ cells/ml.

### Example 2

### Preparation of Cell Support Materials

Support material, which was oriented fibers of type I collagen (Organogenesis, Inc.) was rinsed in sterile, deionized water; placed into a solution of 70% ethanol for 24 hours, then rinsed in sterile, deionized water; and stored in sterile Dulbecco's Phosphate Buffered Saline. A stock solution of fibronectin (Collaborative Research, Waltham, Mass., Cat # 35-4008) was prepared by adding 10 ml of sterile deionized water to a 1 mg vial of fibronectin. This should be done several hours before use to assure that the fibronectin is solubilized. (Note: A laminin stock solution, when used, can be prepared in a similar manner.) The support material was placed into the fibronectin stock solution for 24 hours at 4° C. After incubation, the support material was rinsed in cell-culture medium just prior to use.

### Example 3

### Bioreactor Procedure

The procedure for using the bioreactor for preparing constructs in one embodiment included: rinsing the bioreactor thoroughly with sterile, deionized water taking care to assure that foreign materials was not introduced and avoiding the use of surfactants. The bioreactors was sterilized by autoclaving. Alternatively, it could be sterilized by rinsing with 70% ethanol; but ethylene oxide should not be used. The bioreactor was rinsed again in sterile, deionized water and loaded with support materials, cell suspension, and medium. Air bubbles that were present were removed. The inoculation density depends on the desired results. The preferred density of this embodiment is 1 ml of medium for 1×10⁶ cells on 5 cm² of support surface. Changes to this ratio alter the overall size and cellular thickness of the constructs prepared. The bioreactor was attached to the rotating platform, which was a device using a small variable speed motor (0-100 RPM) and a simple mounting platform. The rotation rate of the bioreactor was set to keep the support material and cells in static suspension as described above. As the bioreactor rotated, it was possible to visualize the elliptical path traced by the included support surfaces (the cells were too small to be seen easily) using oblique illumination from a penlight. As the rate of rotation was increased, the elliptical paths got smaller until the supports appeared to remain stationary relative to the wall of the bioreactor. The rate is important and if the rate of rotation is set too high, an undesirable centrifugal effect may be seen. The bioreactor was placed into an incubator at 37° C. in 95-100% humidity with CO₂. The level of CO₂ depends on the medium chosen, especially the amount of sodium bicarbonate level in the medium. For example, AI-1 medium containing 2.45 mg/ml sodium bicarbonate was used in a 5% CO₂ environment. The final pH of the medium was about ˜7.2;

The culture was periodically assessed to determine 1) the extent of attachment of the cells to the support material, and 2) to observe the contractile ability (i.e. beating) of the attached cells. Assessment was done by either taking a sample of cells from the HARV reactor for microscopic observation or placing a VueLife bag directly on the microscope stage. The results showed that cells became attached to the support materials within 48 hr and were spontaneously beating within 72 hr of inoculation into the bioreactor. The medium was changed at 24 hr to remove any unattached cells. The exchange of medium, addition of compounds, and sampling of the vessels can be carried out through the available access ports. Subsequently, the culture was fed with a complete change of medium at 48 hr intervals. The 3D architecture was observed within the first 6 days of culture. In practice, it is anticipated that bio-active constructs will be assessed using a battery of profiling methods to assure performance and quality. Profiling methods may include, for example: 1) genomic profiling using custom micro-arrays, 2) proteomic profiling using 2-D gel electrophoresis, 3) metabolic profiling using a set of assays to measure enzyme activities and substrate utilization characteristics, 4) morphometric profiling to determine the organization of the component cells and materials, and 5) functional profiling in which contractile strength and frequency are assessed in response to a set of inotropic and chronotropic effectors. The comparison of individual constructs or samples taken from batches of constructs to benchmark criteria, determined, for example, from the same assays performed on isolated tissue fragments.

### Example 4

### The HARV Reactor

This example describes a HARV (High Aspect Ratio Vessel) which is one example of a reaction vessel that may be used for growing cardiac tissue constructs. This vessel may be obtained from Synthecon, Inc. of Friendswood, Tex. The HARV consists of a base, a controller, and a culture vessel. The base holds a small motor linked by a belt drive mechanism to the shaft of the culture vessel mounting. Since the HARV culture vessel is a closed system, the base also contains a small air pump that provides a sterile filtered air and CO₂ mixture from the incubator environment in which the HARV is operated. The controller supplies the variable voltages to drive the motor and air pump. The culture vessel itself is a machined polycarbonate (Lexan) culture dish that is operated in the upright position. Each vessel is comprised of two halves: one portion screws onto the base and contains several small pores that are covered by a silicone membrane (which serves as a gas exchange for the medium); the other portion has a circular recessed area and two luer-locking ports through which cells and media can be accessed. The two halves are bolted together and sealed by means of a compressible O-ring. When assembled and loaded, the HARV can be mounted onto the motorized base and rotated.

The key to the utility of the HARV system is that the culture vessel and the fluid contained therein can approximate a solid body during rotation. When cells, microcarrier beads, or other materials are added to a non-rotating HARV, they sink to the bottom of the vessel at a constant rate (except for a nearly instantaneous initial acceleration due to gravity). This sedimentation rate of a particle is related to the gravitational field, the difference in density between the particles and the medium, the size of the particle, medium viscosity, and other factors. If the HARV is rotated while the materials sediment, they will appear to describe an arc determined by the combination of continual sedimentation and movement with the culture medium. If the rotation rate of the vessel and medium is increased, the particles can be prevented from reaching the bottom of the vessel, and they will describe an elliptical path through the medium relative to the observer. As the rotation rate is increased further, the dimensions of the elliptical path diminish until the particles become essentially motionless relative to the medium. At this point the vessel, medium, and contents approximate a single solid body: Particles (i.e. cells, cells on microcarrier beads, etc.) maintain three dimensional orientations relative to each other and the surrounding medium in a relatively low shear environment. Under these conditions, materials within the vessel are essentially motionless relative to the surrounding fluid. This minimizes damaging fluid shear while allowing the cells the ability to co-localize in a fluid environment.

A variety of cell types have been successfully cultured in HARV bioreactors. These include satellite cells, cells for cell-polymer cartilage implants, small intestinal cells, colon carcinoma cells, ovarian tumor cells, rat heart cells, and rat ventricular cells. These tissue cultures produced masses of cells of a particular cell type. In the case of the rat heart cell the disorganized mixtures of cardiac cells to failed to re-establish organotypic structure within HARV bioreactors. The development of an organotypic structure represents a crucial step in the tissue engineering of cardiac constructs.

It will be readily understood by those persons skilled in the art that the present invention is susceptible to broad utility and application. Many embodiments and adaptations of the present invention other than those herein described, as well as many variations, modifications and equivalent arrangements, will be apparent from or reasonably suggested by the present invention and foregoing description thereof, without departing from the substance or scope of the invention.

Accordingly, while the present invention has been described here in detail in relation to its preferred embodiments, it is to be understood that this disclosure is only illustrative and exemplary of the present invention and is made merely for the purposes of providing a full and enabling disclosure of the invention. The foregoing disclosure is not intended to be construed or to limit the present invention or otherwise to exclude any other such embodiments, adaptations, variations, modifications and equivalent arrangements, the present invention being limited by the claims and the equivalents thereof.

What is claimed is:

1. A cardiac construct comprising cardiac cells organized on a support to provide a three-dimensional cell structure having physiological characteristics of cardiac tissue.

2. The cardiac construct of claim 1, wherein the cardiac cells are mammal cells selected from the group consisting of cardiomyocytes, endocardial cells, cardiac adrenergic cells, cardiac fibroblasts, vascular endothelial cells, smooth muscle cells, cardiac progenitor cells, and stem cells.

3. The cardiac construct of claim 1, wherein the at least one support is selected from the group consisting of a suture, a mesh, a foam, a gel, a ceramic, and an acellularized extra-cellular matrix material.

4. The cardiac construct of claim 1, wherein the at least one extra-cellular support is a suture comprised of a component selected from the group consisting of silk, polypropylene, polyamide, polyester, polyvinylidene, poly dioxanone, nylon, linen, cotton, plain gut, chromic gut, poliglecaprone, polyglactin, polylactide, collagen, naturally occurring protein, and combinations thereof.

5. A method for the augmentation of cardiac tissue in a patient comprising the steps of: providing human cardiac construct comprising cardiac cells on a support wherein the cardiac cells are organized on the support to provide a three-dimensional cell structure having physiological characteristics of cardiac tissue for implantation into cardiac tissue of a patient.

6. The method of claim 5, further comprising implanting a compound in the patient selected from the group consisting of compounds that enhance vascularization, compounds that enhance cell survival, compounds that enhance cell proliferation, compounds that enhance cell differentiation, compounds that enhance tissue differentiation, compounds that inhibit fibrosis, compounds that inhibit inflammation and compounds that inhibit tumorigenesis.

7. A method for the testing of cardiac tissue comprising the steps of:
exposing a cardiac construct having cardiac cells organized on a support to provide a three-dimensional cell structure having physiological characteristics of cardiac tissue to a test compound; and
observing a response of said cardiac construct to the test compound.

8. The method of claim 7, further comprising attaching the cardiac construct to at least one strain gage, wherein the observed response is observing a change in the strain gage.

9. The method of claim 7, wherein the test compound is selected from the group consisting of compounds that have a chronotropic effect and compounds that have an inotropic effect.

10. The method of claim 7, further comprising the step of adding a dye to the cardiac construct.

11. The method of claim 10, wherein the response is observed by a detection instrument sensitive to the dye and where in the test compounds are selected from the group consisting of compounds that affect conduction, compounds that affect gene expression, compounds that affect cellular organization, and compounds that affect multicellular organization.

12. A bio-active cardiac construct kit comprising;
a bio-active cardiac construct having cardiac cells organized on a support to provide a three-dimensional cell structure having physiological characteristics of cardiac tissue; and
a housing surrounding the bio-active cardiac construct.
